(19) United States
(12) Patent Application Publication
(10) Pub. No.: US 2022/0011200 A1
(43) Pub. Date: Jan. 13, 2022

(54) METHODS FOR LOADING A DIGITAL PCR CHIP

(71) Applicants: Yan Wang, San Diego, CA (US); Raymond Yang, San Diego, CA (US)

(72) Inventors: Yan Wang, San Diego, CA (US); Raymond Yang, San Diego, CA (US)

(21) Appl. No.: 16/924,204

(22) Filed: Jul. 9, 2020

(51) Int. Cl. G01N 1/28; C12Q 1/6806; C12Q 1/686

(52) U.S. Cl. CPC G01N 1/2813; C12Q 1/686; C12Q 1/6806

(57) ABSTRACT

Disclosed are methods and devices for loading a liquid sample into an array substrate with a plurality of compartments, for example, a dPCR chip. The method uses a loading device having a single flat surface to efficiently load a liquid sample into a plurality of compartments in the array substrate.

### FIELD OF THE INVENTION

This invention belongs to the field of biotechnology, especially relates to methods for loading a liquid sample to a microchip for performing digital polymerase chain reactions.

### BACKGROUND OF THE INVENTION

Polymerase chain reaction (PCR) is a method that uses DNA polymerization reaction to generate millions and billions of nucleic acids of interest. It performs repeated cycles of denaturing of double-stranded DNA, annealing of primers to target sequences, and extending of primers to generate copies of target sequences. PCR is an indispensable technique in molecular biology that is widely used to detect, identify, obtain and quantitate a DNA/RNA sequence of interest.

Digital PCR (dPCR) is a refinement of PCR technologies that allows absolute quantification of nucleic acid strands and sensitive detection of rare DNA targets. The dPCR improves upon conventional PCR by separating one PCR sample into a large number of partitions to perform a large number of PCRs in parallel. The PCR sample is partitioned such that each partition on average contains no more than one target nucleic acid molecule, that is, each partition approximately contains either 1 or 0 target nucleic acid molecule. By detecting partitions with positive amplifications, digital PCR enables absolute quantification of target nucleic acids in a sample. The one commonly used dPCR uses a chip-based format where a PCR sample is partitioned into a large number of micro-wells in a dPCR chip. A typical dPCR chip has a size of about 1 cm×1 cm and contains 20,000 or more micro-wells. The first step of a dPCR experiment is to load a liquid solution containing a nucleic acid sample and dPCR components into micro-wells of a dPCR chip. The loading method needs to be able to efficiently and evenly load sample volumes into majority of micro-wells. We have designed and developed a sample loader that is a single-use consumable consumed in every dPCR experiment. It is low cost, easy to make and can be used independent of any instruments.

### SUMMARY OF THE INVENTION

According to some embodiments of the invention, there provides a simple loading device for loading a liquid sample into an article with a plurality of compartments, for example, a dPCR chip, wherein the loading device comprises a single flat surface for holding a liquid sample and distributing the liquid sample into the plurality of compartments of the article.

According to some embodiments of the invention, a method is provided for loading a liquid sample to a digital PCR chip with a plurality of compartments, comprising the steps of: 1) placing a liquid sample onto a flat surface of a loading device; 2) sandwiching the liquid sample between the loading device and a digital PCR chip at an angel between 0 to 90 degree; and 3) moving the loading device relative to the digital PCR chip to distribute the liquid sample into the plurality of compartments of the digital PCR chip.

According to some embodiments of the invention, the flat surface of the loading device is non-absorbent to water.

According to some embodiments of the invention, the flat surface of the loading device is treated to be hydrophilic.

According to some embodiments of the invention, the hydrophilic flat surface of the loading device has a hydrophobic line to limit the spread of the liquid sample.

According to some embodiments of the invention, the loading device can be moved relative to the digital PCR chip in a circular or linear direction.

According to some embodiments of the invention, the loading device comprises a flat sheet that matches to the size and shape of the digital PCR chip to be loaded.

According to some embodiments of the invention, the loading device comprises a loading part with a flat surface and a handle.

According to some embodiments of the invention, the loading device is composed of a material from the group consisting of polycarbonate, polyethylene, polystyrene, acrylic, polylactic acid, polypropylene, polyolefin, and polyvinyl chloride.

According to some embodiments of the invention, the liquid sample comprises one or more components selected from a group consisting of BSA, Triton X-100, Tween-20, NP-40, TMAC, Ethanol, PEG, and KCl.

### BRIEF DESCRIPTION OF THE DRAWINGS

FIG. 1 shows an example of a dPCR chip with a plurality of micro-wells.

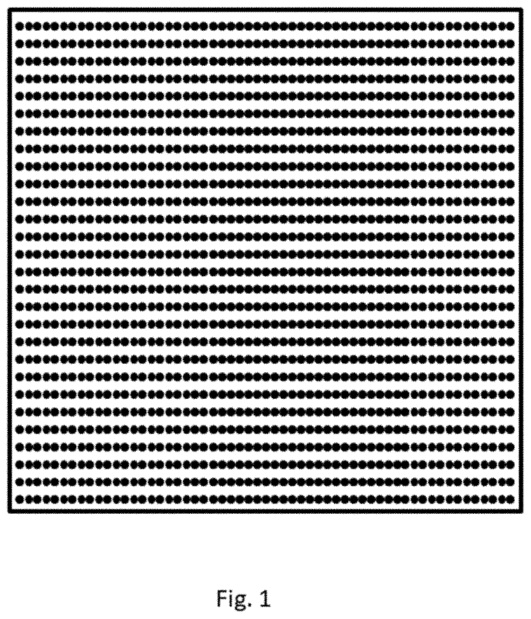

FIG. 2 shows an exemplary embodiment of a loading device as a flat sheet that matches to the size and shape of the dPCR chip to be loaded.

FIG. 3 shows a method of loading a liquid sample into a plurality of micro-wells of a dPCR chip using an exemplary loading device of FIG. 2.

FIG. 4 shows two exemplary embodiments of loading devices having a flat surface for holding a liquid sample and a handle.

FIG. 5 shows a method of loading a liquid sample into a plurality of micro-wells of a dPCR chip using an exemplary loading device of FIG. 4.

FIG. 6A shows a dPCR experiment result using a loading device with a handle of the invention.

FIG. 6B shows a dPCR experiment result using a parafilm sheet as a loading device.

### DETAILED DESCRIPTION

### Definitions

Unless otherwise defined, all technical and scientific terms used herein have the same meaning as commonly understood by one of the ordinary skills in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The present invention relates to methods and devices for loading a liquid sample into a large number of compartments in an array substrate so that a large quantity of detection reactions can be performed in parallel. In particular, it relates to methods and devices for partitioning a sample into a large quantity of very small sample volumes, for example, less than 1 nanoliter, less than 100 picoliters, or less than 10 picoliters.

According to some embodiments of the invention, the liquid sample may comprise any suitable biological samples of interest, including, but not limited to, samples containing nucleic acids such as RNA or DNA, proteins, polypeptides, lipids, polysaccharides or any combinations thereof. The liquid sample may also contain targets such as tumor cells, viral particles, bacterial cells, or other biological substances. The liquid sample comprises a biological sample with targets to be detected and a reaction buffer containing components needed for performing the detection reaction. In addition, it contains one or more chemicals to facilitate the entry into the small compartments in the array article, including, but not limited to, BSA, Triton X-100, Tween-20, NP-40, TMAC, Ethanol, PEG, and KCl.

According to some embodiments of the invention, the detection reaction may be any reaction suitable for detecting biological targets of interest, including, but not limited to, PCRs, antigen-antibody binding reactions, ligand-receptor binding reactions, and enzyme-substrate reactions. Suitable PCR methods include, for example, conventional PCR, quantitative PCR and dPCR. The invented method is particularly suitable for use with array-format dPCR where a PCR sample is partitioned into a large number of small reaction sites with volumes usually equal or less than 1 nanoliter and PCR amplifications are performed in individual reaction sites.

According to some embodiments of the invention, the array substrate can be of any desired shape, for example, rectangular shape, circular shape or hexagonal shape. The most common shape of the array substrate is a rectangular one such as a rectangular plate or chip. The substrate is composed of solid materials including, for example, glass, metal, plastics, and ceramics. Preferably, the inside surface of the compartments in a substrate is made to be hydrophilic and the surface area of the substrate outside the compartments is made to be hydrophobic. In this way, the liquid solution can be easily drawn to the inside of the compartments, and cross-talk between neighboring compartments can be minimized. The compartments in the substrate can take various forms, for example, spots, micro-wells, chambers, through-holes, indentations and cavities. The plurality of compartments can be arranged as an ordered or random array in the substrate. The number of compartments in a substrate depends on the throughput requirement of a particular assay. A substrate may contain, for example, 100, 1000, 10000, 20000, 30000 or more compartments. The volume of a compartment can be, for example, several nanoliters, equal or less than a nanoliter, equal or less than 10 picoliters. In a substrate of a particular volume, the bigger the number of compartments in the substrate, the smaller the volume of each compartment. For example, a dPCR chip has about 20,000 compartments in a plate with the size of 1 cm×1 cm×0.3 mm (length×width×height) and each compartment has a volume of less than 2 nanoliters.

In an embodiment of the invention, a method is provided for loading a liquid sample to a digital PCR chip with a plurality of compartments, comprising the steps of: 1) spreading a liquid sample onto a flat surface of a loading device; 2) sandwiching the liquid sample between the loading device and a digital PCR chip at an angel between 0 to 90 degree; and 3) moving the loading device relative to the digital PCR chip to distribute the liquid sample into the plurality of compartments of the digital PCR chip.

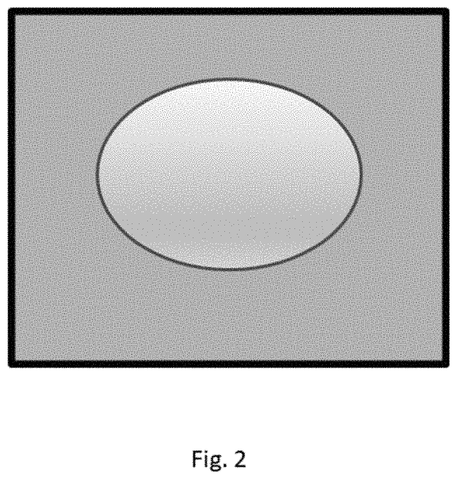

The loading device comprises a loading portion having a flat surface for holding a liquid sample. In some embodiment, the loading portion of the loading device is a thin flexible sheet having the size and shape that matches to those of the dPCR chip to be loaded. It can be composed of polymer materials, including, but not limited to, polycarbonate, polyethylene, polystyrene, acrylic, polylactic acid, polypropylene, polyolefin, and polyvinyl chloride. It is made to be thin to provide enough flexibility that is needed to facilitate the delivery of liquid samples into small compartments on the dPCR chip. The thickness of the loading portion can be, for example, equal or less than 0.5 mm, equal or less than 1 mm, equal or less than 2 mm, or equal or less than 5 mm. For example, a parafilm cut into a sheet of the size and shape of a dPCR chip can be used as a loading device. FIG. 2 shows a loading device comprising a thin flat sheet. The liquid sample is deposited and spread in the middle of the loading device. In some embodiments, the loading device is made of or coated with a material that is non-absorbent to water. This can help prevent sample loss due to the absorbance of the liquid sample to the loading device. In some embodiments, the non-absorbent surface of the loading portion is further treated to be hydrophilic, which can help spread the liquid sample on the loading device and facilitate even distribution of the liquid sample into more compartments. Methods to make hydrophilic coating onto a surface is well known to one with ordinary skills in the field, including, for example, plasma treatment, high energy radiation, grafting method, and chemical bonding method.

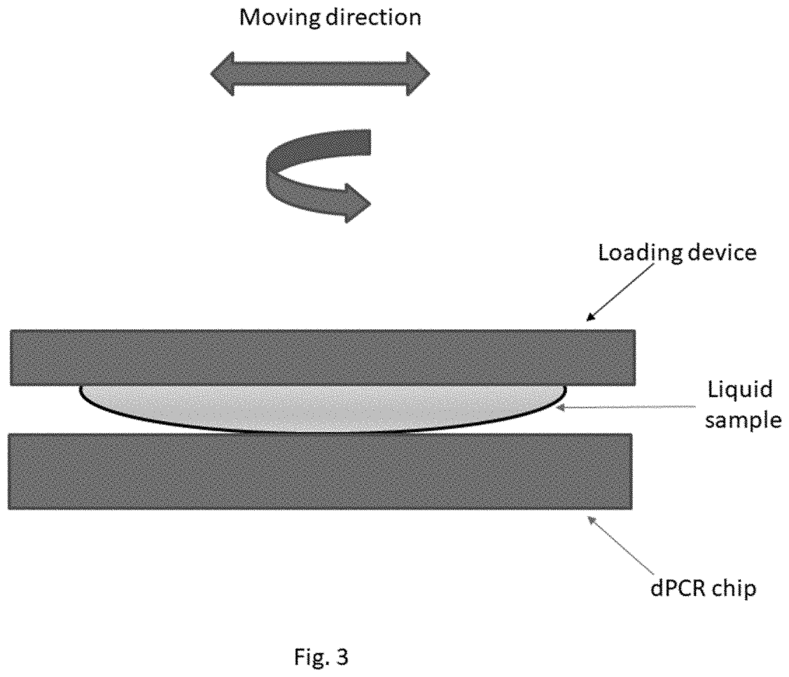

To load a liquid sample into a dPCR chip, the liquid sample is first deposited and spread onto the flat surface of the loading device as shown in FIG. 2. A dPCR chip is placed upside down onto the liquid sample, making a three-layer sandwich with the dPCR chip on the top, the liquid sample in the middle, and the loading device at the bottom. The three-layer sandwich is flipped vertically for 180 degree, resulting in a sandwich with the loading device on the top, the liquid sample in the middle, and the dPCR chip at the bottom (see FIG. 3). The loading device is moved relative to the dPCR chip to facilitate delivering of liquid sample into the plurality of compartments of the dPCR chip. The loading device can be moved in circular or back-and-forth linear mode until all the solution is delivered into the compartments of the dPCR chip. For some instances, a uni-directional movement is sufficient to load all liquid into the micro-wells. Alternatively, the liquid sample is spread onto the flat surface of the loading device, and the loading device with the liquid sample is directly flipped over onto a dPCR chip to make a sandwich with the loading device on the top, the liquid sample in the middle, and the dPCR chip at the bottom. The loading device on the top of the sandwich is then moved around to help deliver the liquid sample into the compartments of the dPCR chip.

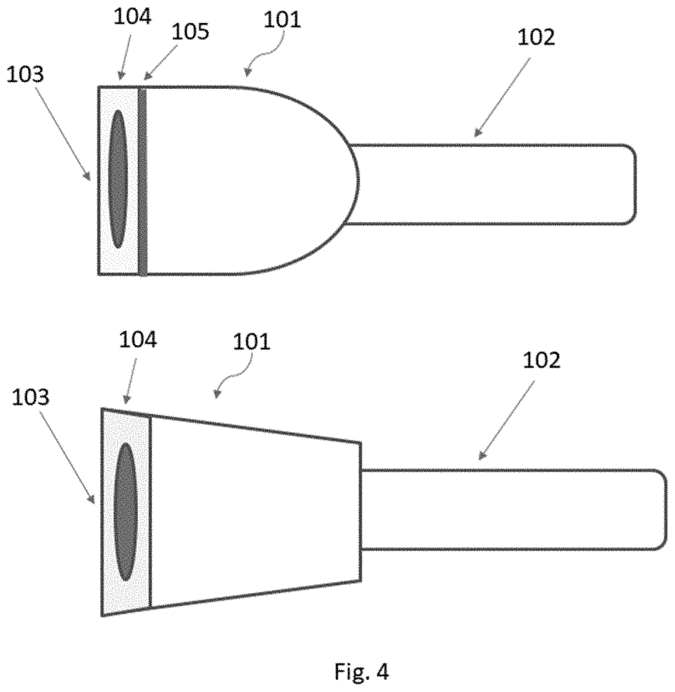

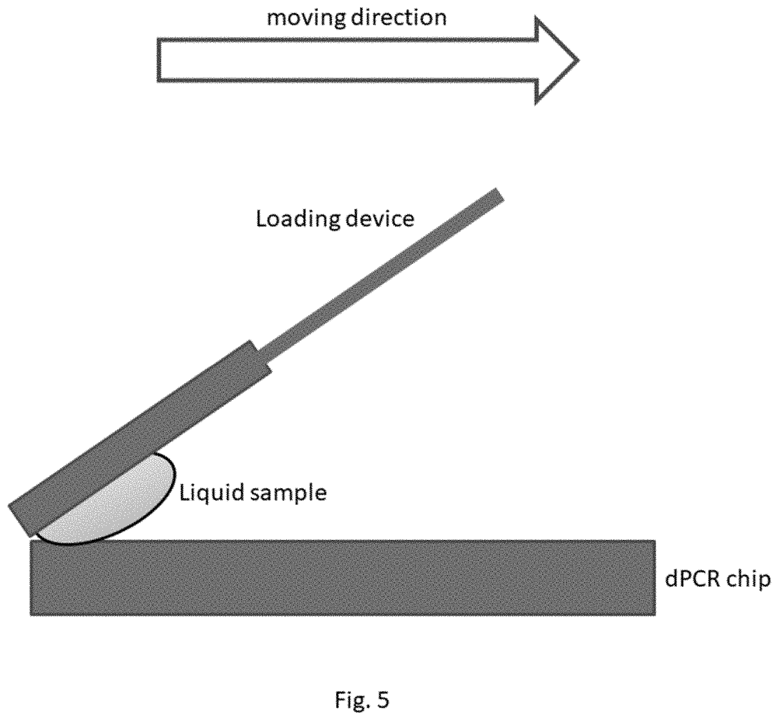

In some embodiments, the loading device comprises a loading portion having a flat surface for holding a liquid sample and a handle for user to grab. FIG. 4 shows two exemplary loading devices having a loading portion <101> and a handle <102>. The loading portion of the loading device has a flat surface and a straight front edge <103>. The length of the front edge is similar to the width of the dPCR chip. The liquid sample is deposited onto a loading area <104> close to the front edge. The loading portion is made of or coated with a material that is non-absorbent to water and it can be further treated to make the loading area hydrophilic. In addition, a hydrophobic line <105> can be added at the border of the loading area. In this way, the liquid sample can be easily spread within the loading area, but not outside the boundary of the loading area. The loading portion can be composed of polymer materials, including, but not limited to, polycarbonate, polyethylene, polystyrene, acrylic, polylactic acid, polypropylene, polyolefin, and polyvinyl chloride. The loading portion can be made of various shapes, for example, triangle, rectangle, semicircle or trapezoid. FIG. 5 shows a method to load a liquid sample using the loading device with a handle. The liquid sample is first deposited and spread onto the loading area of the loading device. The loading device is turned over, allowing the liquid sample to contact the dPCR chip at an angle between 0-90 degree. The angle between the loading device and the dPCR chip can be, for example, 75-85 degree, 65-75 degree, 55-65 degree, or 45-55 degree. The loading device is then moved slowly along the dPCR chip to deliver the liquid sample into the compartments of the dPCR chip. The moving speed of the loading device can be, for example, 40 sec/cm, 20 sec/cm, 10 sec/cm, 5 sec/cm, or 2 sec/cm.

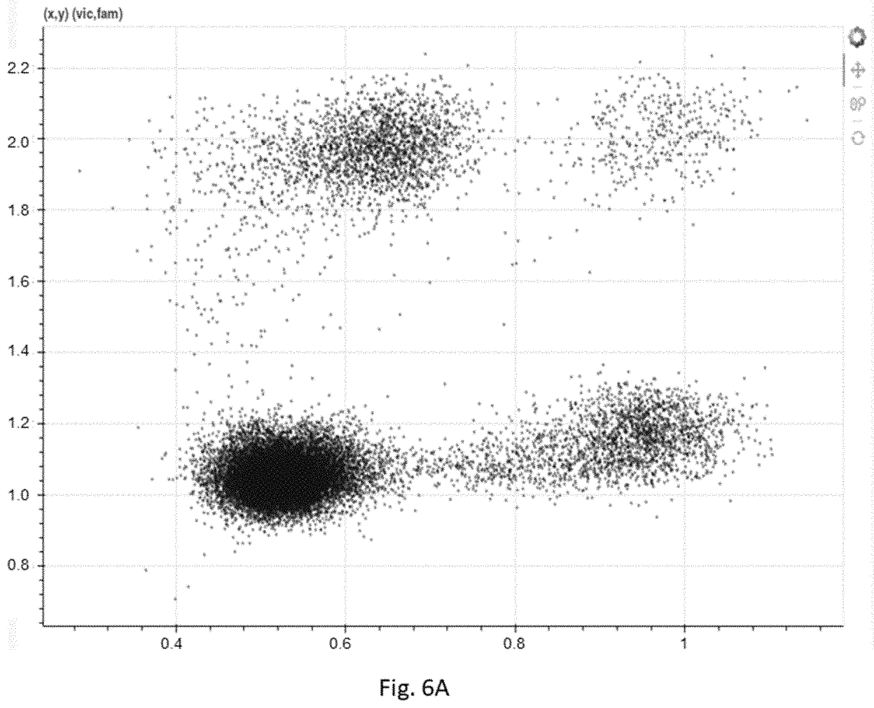

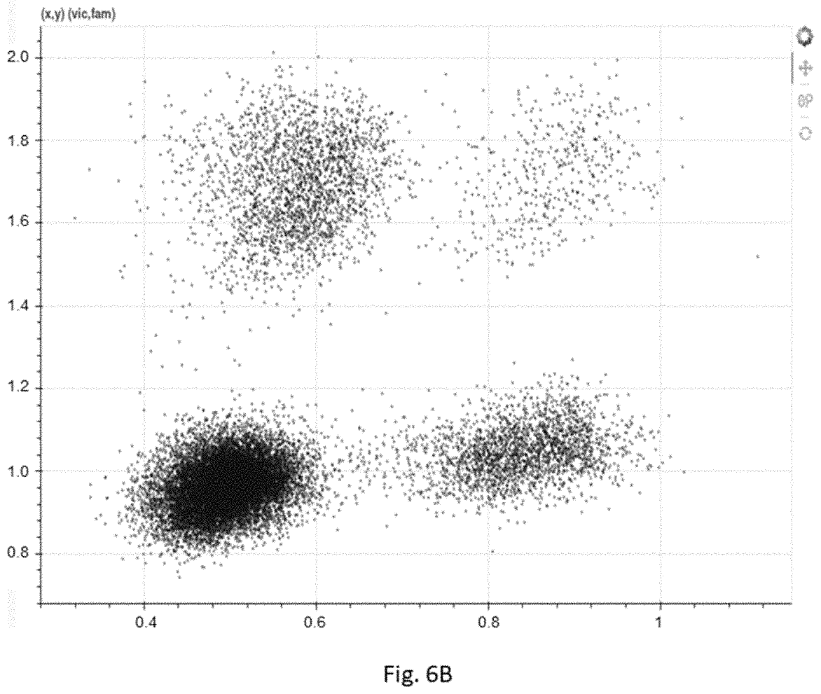

Using the loading method of the invention, we are able to generate digital PCR results meeting our expectations. As shown in FIGS. 6A & 6B, the loading method of the invention is used for a HER2 (FAM)/RNAse P (VIC) copy number assay in a digital PCR analysis on human genomic DNA. Four clusters (no signal cluster, FAM signal only cluster, VIC signal only cluster, and FAM/VIC double signal cluster) were generated with good separation and enough wells were loaded with liquid sample, suggesting that the loading method worked properly. The dPCR results using the loading device with a handle is shown in FIG. 6A. The dPCR result using a parafilm sheet as a loading device is shown in FIG. 6B. The dPCR results are quite similar in these methods. The loading device of the invention uses a single flat surface to hold and distribute liquid samples. The manufacture process of making the loading device of the invention is much easier and less error prone than that of other commercially available loading devices, allowing significant reduction of the production cost. The loading process is simple and straightforward, which can be completed within 10 seconds without need of an expensive loading machine. With simpler design, reduced cost and comparable end results, the loading method of the invention provides a significant advantage over the currently available loading methods.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for loading a liquid sample to a digital PCR chip with a plurality of compartments, comprising the steps of:
1) spreading a liquid sample onto a flat surface of a loading device, wherein the loading device uses a single flat surface for holding and delivering the liquid sample;
2) sandwiching the liquid sample between the loading device and a digital PCR chip at an angel between 0 to 90 degree; and
3) moving the loading device relative to the digital PCR chip to distribute the liquid sample into the plurality of compartments of the digital PCR chip.

2. The method of claim 1, wherein the flat surface of the loading device is non-absorbent to water.

3. The method of claim 1, wherein the flat surface of the loading device is treated to be hydrophilic.

4. The method of claim 3, wherein the hydrophilic flat surface of the loading device has a hydrophobic line to limit the spread of the liquid sample.

5. The method of claim 1, wherein the loading device can be moved relative to the digital PCR chip in a circular or unidirectional mode.

6. The method of claim 1, wherein the loading device comprises a flat sheet.

7. The method of claim 1, wherein the loading device comprises a loading part with a flat surface and a handle.

8. The method of claim 1, wherein the loading device is composed of a material of the group consisting of polycarbonate, polyethylene, polystyrene, acrylic, polylactic acid, polypropylene, polyolefins, and polyvinyl chloride

9. The method of claim 1, wherein the liquid sample comprises one or more components selected from a group consisting of BSA, Triton X-100, Tween-20, NP-40, TMAC, Ethanol, PEG, and KCl.
